(19) United States
(12) Patent Application Publication
(10) Pub. No.: US 2022/0151531 A1
(43) Pub. Date: May 19, 2022

(54) HEART FAILURE PREDICTOR AND HEART FAILURE PREDICTING METHOD

(71) Applicants: Inventec (Pudong) Technology Corporation, Shanghai (CN); INVENTEC CORPORATION, Taipei (TW)

(72) Inventors: Davide Burba, Taipei (TW); Trista Pei-Chun CHEN, Taipei (TW)

(21) Appl. No.: 17/165,063

(22) Filed: Feb. 2, 2021

(30) Foreign Application Priority Data
Nov. 18, 2020 (CN) 202011293024.2

(51) Int. Cl. A61B 5/319; A61B 5/349; A61B 5/341; A61B 5/00; G16H 50/20

(52) U.S. Cl. CPC A61B 5/319; A61B 5/349; G16H 50/20; A61B 5/7267; A61B 5/341

(57) ABSTRACT

A method of heart failure prediction comprising obtaining a raw electrocardiogram (ECG) signal by a sensor, generating a clean ECG signal according to the raw ECG signal by a pre-processing circuit, performing, by a feature extraction circuit, a principal component decomposition and a heart rate feature analysis according to the clean ECG signal to generate a feature vector with a plurality of features, and generating a prediction to indicate whether the heart failure will happen in a specified period according to the feature vector by a predicting model circuit.

### CROSS-REFERENCE TO RELATED APPLICATIONS

This non-provisional application claims priority under 35 U.S.C. § 119(a) on Patent Application No(s). 202011293024.2 filed in China on Nov. 18, 2020, the entire contents of which are hereby incorporated by reference.

### BACKGROUND

### 1. Technical Field

This disclosure relates to the prediction of heart failure, and more particular to a heart failure predictor and heart failure predicting method.

### 2. Related Art

Congestive Heart Failure (CHF) is a highly lethal syndrome whose symptoms and signs are caused by cardiac dysfunction. Healthcare expenditure on CHF in developed countries consumes 1-2% of the total health care budget. For these reasons, improving the comprehension of the CHF dynamics may have a strong social and managerial impact.

It is well known that the electrocardiogram (ECG) test can be used to extract predictive features to assess individuals' risk to develop CHF.

The ECG signal is a cyclic signal which quantifies the electrical activity of the heart. Each heart cycle can be decomposed in the sum of five waves, namely P, Q, R, S, T waves. Along with the shape of each heart cycle, another important feature of the ECG is the duration of the heart cycles. These are measured based on the length of the RR-intervals, i.e. the distance of consecutive R-peaks, and they are typically summarized via variables quantifying the Heart Rate (HR) and the Heart Rate Variability (HRV) of the individual.

Machine Learning (ML) is a field of computer science which has proven extremely successful at modelling complex non-linear relations between a set of predictive variables and various outcomes. ML has been applied to predict the development of CHF and other outcomes such as death based on ECG signals. However, since ML methods estimate complex non-linear relations between high-dimensional feature, they are typically hard to interpret.

Further, current methods take a long time the collect data for predicting CHF, and the possibility of predicting the development of CHF with fully interpretable ML models by using only a small amount of ECG data has not yet been explored.

### SUMMARY

Accordingly, the present disclosure provides a heart failure predictor and a heart failure predicting method with high measure efficiency and interpretability.

According to one or more embodiment of this disclosure, a heart failure predictor comprises: a pre-processing circuit configured to electrically connect to an external sensor to receive a raw electrocardiogram (ECG) signal and configured to filter a noise of the raw ECG signal to generate a clean ECG signal; a feature extraction circuit electrically connecting to the pre-processing circuit, wherein the feature extraction circuit calculates a plurality of heart rate features according to the clean ECG signal, generates a plurality of shape features according to a plurality of principal component waveforms and the clean ECG signal, and concatenates the plurality of heart rate features and shape features to generate a feature vector; and a prediction model circuit electrically connecting to the feature extraction circuit and generating a prediction according to the feature vector; wherein the prediction is configured to indicate whether the heart failure will happen in a specified period.

According to one or more embodiment of this disclosure, a heart failure predicting method comprising: obtaining a raw electrocardiogram (ECG) signal by a sensor; generating a clean ECG signal according to the raw ECG signal by a pre-processing circuit; performing, by a feature extraction circuit, a principal component decomposition and a heart rate feature analysis according to the clean ECG signal to generate a feature vector with a plurality of features; and generating a prediction to indicate whether the heart failure will happen in a specified period according to the feature vector by a predicting model circuit.

In view of the above description, the heart failure predictor and a heart failure predicting method proposed in the present disclosure uses the “shape” feature, that is extracted from a time-series data, to predict the CHF together with HR and HRV. The present disclosure may generate an interpretable feature vector so that a doctor may explain the cause of heart failure to the patient based on the clinical symptoms corresponding to the feature vector. Because the feature extraction circuit of the present disclosure uses the principal component analysis technique, the feature vector includes the shape feature of the ECG signal. Using principal component analysis also improves the accuracy of the present disclosure in predicting heart failure. The present disclosure can generate a prediction result for the long-term future (several months to several years) by collecting one's ECG signal for only a short period of time (for example, 30 seconds).

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 is a block diagram of a heart failure predictor according to an embodiment of the present disclosure;

FIG. 2 is a block diagram of a pre-processing circuit;

FIG. 3A and FIG. 3B respectively show the ECG signal charts before and after the operations of the band-pass filter of the pre-processing circuit;

FIG. 4 is a block diagram of a feature extraction circuit;

FIG. 5 is a flowchart of a method of heart failure prediction according to an embodiment of the present disclosure; and

FIG. 6 is a detailed flowchart of “principal component analysis” in step S3 of FIG. 5.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawings.

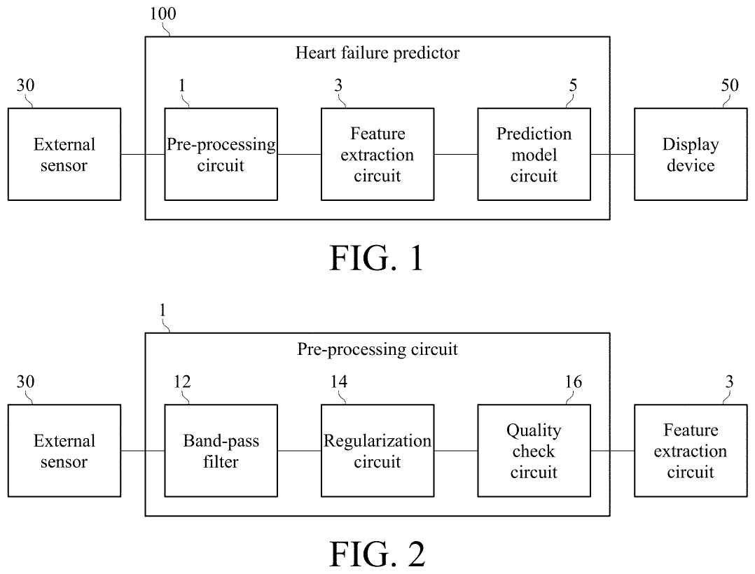

Please refer to FIG. 1 which illustrates a block diagram of a heart failure predictor 100 according to an embodiment of the present disclosure. The heart failure predictor 100 comprises a pre-processing circuit 1, a feature extraction circuit 3, and a prediction model circuit 5.

The pre-processing circuit 1 is configured to electrically connect to an external sensor 30, such as Holter monitor, to receive a raw electrocardiogram (ECG) signal generated by the external sensor 30. Comparing to the conventional prediction method that takes a long time (for example, about 24 hours) to collect the user's ECG signal, the pre-processing circuit 1 of the present disclosure only takes a short period of time (for example, about 30 seconds) to collect the user's ECG signal.

Please refer to FIG. 2, which illustrates a block diagram of a pre-processing circuit 1. In an embodiment, the pre-processing circuit 1 comprises a band-pass filter 12, a regularization circuit 14 and a quality check circuit 16.

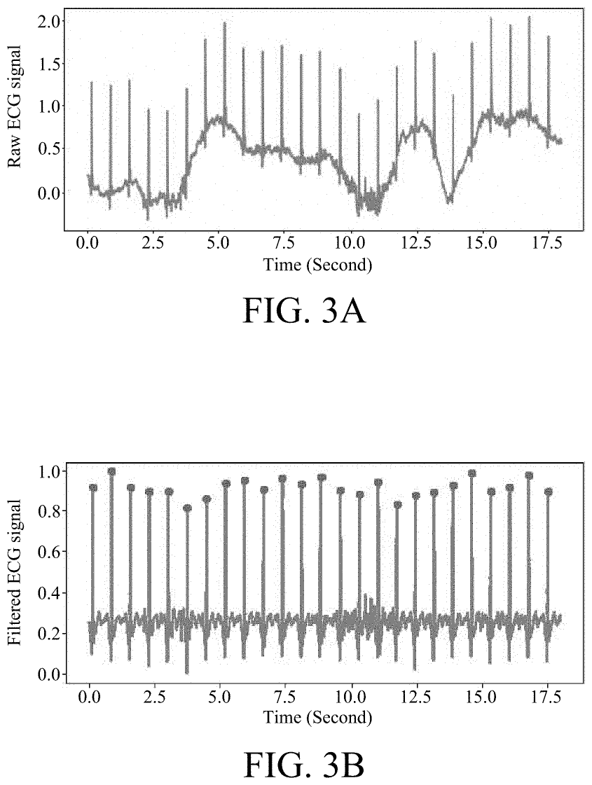

The band-pass filter 12 is configured to electrically connect to the external sensor 30 of FIG. 1. In an embodiment, the band-pass filter 12 is configured to filter the noise of the raw ECG signal and correct the baseline wander of the raw ECG signal. The ECG signal before the correction is shown in FIG. 3A, and the ECG signal after the correction is shown in FIG. 3B. The corrected ECG signal is sent to the regularization circuit 14.

The regularization circuit 14 electrically connects to the band-pass filter 12. In an embodiment, the regularization circuit 14 is configured to delimit the minimum of every cycle of the corrected ECG signal as zero and delimit the maximum of every cycle of the corrected ECG signal as one. In other words, the regularization circuit 14 scales the signal in a [0, 1] interval without changing the original distribution of the signal.

The quality check circuit 16 electrically connects to the regularization circuit 14. The quality check circuit 16 is configured to estimate the quality of ECG signal processed by the above steps. Specifically, the quality check circuit 16 calculates a signal quality index (SQI) of the ECG signal. The signal segment whose SQI is higher than a threshold will be preserved to be inputted to the feature extraction circuit 3. The signal segment whose SQI is lower than the threshold will be dropped.

The present disclosure does not limit the implementation of the quality check circuit 16. In an embodiment, the external sensor 30 connecting to the heart failure predictor 100 further comprises an environment sensor such as an accelerometer or a light sensor. The environment sensor is configured to sense the state of the environment near the user to generate a corrected reference signal, and the band-pass filter 12 fix the raw ECG signal according to the corrected reference signal. The quality check circuit 16 calculates the standard range of multiple physiological values in each cycle and determines a difference value between the physiological value of each cycle and the standard range according to the corrected ECG signal, and then calculates the SQI according to these difference values. For example, the standard range is the average of physiological values. For another example, the standard range is a confidence interval of a distribution model established according to these physiological values. An example of calculating the SQI according to these difference values is that the quality check circuit 16 calculates a ratio of the number of difference values less than a threshold to the total number of the difference values, and takes the ratio as the SQI.

Another example of calculating the SQI according to these difference values is that the quality check circuit 16 calculates a correlation according to each of difference values and calculates the SQI according to the correlation and the total number of the cycle signals.

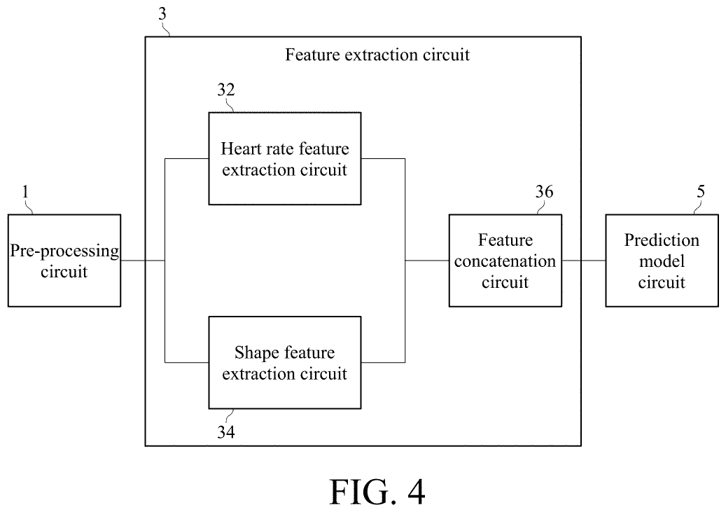

The feature extraction circuit 3 electrically connects to the pre-processing circuit 1 to receive the clean ECG signal processed by the pre-processing circuit 1. FIG. 4 is a block diagram of the feature extraction circuit 3. The feature extraction circuit 3 comprises a heart rate feature extraction circuit 32, a shape feature extraction circuit 34 and a feature concatenation circuit 36. The clean ECG signal is inputted to both the heart rate feature extraction circuit 32 and the shape feature extraction circuit 34. After these two circuits 32 and 34 perform feature extraction respectively, the feature concatenation circuit 36 concatenates their outputs and sends the prediction model circuit 5 the result.

The feature extraction circuit 3 is configured to extract a plurality of important features from the ECG signal and output a multidimensional feature vector. In an embodiment, this feature vector has 14 dimensions, wherein the heart rate feature extraction circuit 32 performs computations with the clean ECG signal to output data of 10 dimensions, and the shape feature extraction circuit 34 performs computations with the clean ECG signal to output data of the remaining 4 dimensions. The above-mentioned dimension values are exemplary and are not used to limit the present disclosure.

The 10-dimensional data outputted by the heart rate extraction circuit 32 and the type of each dimension are listed as the table below, wherein HR refers to heart rate and HRV refers to heart rate variability, and R-R interval refers to a distance between two adjacent peaks in the ECG signal, and this distance may be converted into a heart rate.

Feature Name Description Type MEAN_HR MRRI SDNN HR

| Feature Name | Description | Type |
|---|---|---|
| MEAN_HR | Average of HR | HR |
| MRRI | Average of R-R interval | HR |
| SDNN | Standard deviation of all normal to normal | HRV |
| | R-R intervals | |
| RMSSD | Root-Mean Square of the Successive R-R | HRV |
| | interval differences | |
| NRMSSD | RMSSD normalized to MRRI | HRV |
| SDSD | Standard deviation of the successive R-R | HRV |
| | interval differences | |
| PNN50 | Percentage of successive R-R interval | HRV |
| | differences that differ by more than 50 ms | |
| PNN20 | Percentage of successive R-R interval | HRV |
| | differences that differ by more than 20 ms | |
| SD1/SD2 | Features derived from the Poincaré analysis | HRV |
| shannon_entropy | Shannon Entropy of R-R intervals time series | HRV |

The calculation methods of the features in the above table are shown in the following equations 1 to 9.

$$\begin{matrix} MEAN\_HR = \frac{{\sum }_{i = 1}^{K} \frac{60}{RR interval [ t ]}}{K} & Equation 1 \\ MRRI = \frac{{\sum }_{i = 1}^{K} RR interval [ i ]}{K} & Equation 2 \\ SDNN = \frac{{\sum }_{i = 1}^{K} {( RR interval [ i ] - MRRI )}^{2}}{K - 1} & Equation 3 \\ RMSSD = \sqrt{\frac{{\sum }_{i = 1}^{K - 1} {( RR interval [ i + 1 ] - RR interval [ i ] )}^{2}}{K - 1}} & Equation 4 \\ NRMSSD = \frac{R M S S D}{MRRI} & Equation 5 \\ SDSD = \sqrt{\frac{{\sum }_{i = 1}^{K - 1} {( RR diff [ i ] - \overline{RR diff} )}^{2}}{K - 1}} & Equation 6 \\ wherein RR diff [ i ] = RR interval [ i + 1 ] - RR interval [ i ] , and \overline{RR diff} = \frac{{\sum }_{i = 1}^{K - 1} RR diff [ i ]}{K - 1} & \\ PNN 50 = \frac{{\sum }_{i = 1}^{K - 1} I ( \vert RR diff [ i ] \vert > 0.05 )}{K - 1} & Equation 7 \\ PNN 20 = \frac{{\sum }_{i = 1}^{K - 1} I ( \vert RR diff [ i ] \vert > 0.02 )}{K - 1} & Equation 8 \\ SD 1 / SD 2 = \frac{σ ( \{ a [ 1 ] , … , a [ K - 1 ] \} )}{σ ( \{ b [ 1 ] , … , b [ K - 1 ] )} ; & Equation 9\end{matrix}$$

wherein a[i] is the projection of x[i] in x=−y, b[i] is the projection of x[i] in x=y, and x[i]=[RR interval[i], RR interval[i+1]]^{T}

In an embodiment, based on equation 10, the shape feature extraction circuit 34 calculates the remaining 4-dimensional data, that is, sⱼ in equation 10, according to the clean ECG signal, the given reference heart cycle μ(t), and a set of given principal component waveforms. There are four waveforms in the set of principal component waveforms, so j=1, 2, 3, 4.

C(t)≈μt(t)+Σⱼ₌₁^{K}sⱼPCⱼ(t) Equation 10:

Specifically, the shape feature extraction circuit 34 calculates the average heart cycle C(t) according to the clean ECG signal, subtracts the given reference heartbeat cycle μ(t) from the average heartbeat cycle C(t), and projects the difference onto 4 principal component waveforms PCⱼ(t) to obtain projections of respective dimensions sⱼ. The symbol K in the equation 10 indicates the number of principal component waveforms. The above process is so-called principal component analysis (PCA). Given a set of functional data and a number of basis functions, the PCA decomposition finds the data projection in terms of basis functions and corresponding coefficients which maximizes the explained variability, therefore allowing for a minimal representation.

Each principal component waveform is equivalent to a “shape's description” of ECG signal. Therefore, these principal component are interpretable. In practice, doctors may use the clinical symptoms corresponding to these principal component waveforms to clearly show the cause of the heart failure disease to the patient. In addition, the coefficient sⱼ corresponding to each principal component waveform may be used as a basis for doctors to judge the importance of the principal component waveform.

In practice, the shape feature extraction circuit 34 collects a large amount of ECG data in advance and performs eigenanalysis according these data to obtain the plurality of principal component waveforms.

The feature concatenation circuit 36 electrically connects to the heart rate feature extraction circuit 32 and the shape feature extraction circuit 34. The feature concatenation circuit 36 integrates the multidimensional data (10-dimensional data in this embodiment) from the heart rate feature extraction circuit 32 and the multidimensional data (4-dimensional data in this embodiment) from the shape feature extraction circuit 34. The feature concatenation circuit 36 scales the data range and then output a multidimensional feature vector (14-dimension in this embodiment) to the model prediction circuit 5.

The prediction model circuit 5 electrically connects to the feature extraction circuit 3 to receive the multidimensional feature vector. The prediction model circuit 5 is further configured to electrically connect to a display device 50 to show its prediction result. The prediction model circuit 5 inputs the multidimensional feature vector to a prediction model to generate said prediction result. The prediction model is basically a linear model which generates the prediction result according to the multidimensional feature vector

In an embodiment, the prediction model circuit 5 comprises at least one prediction model. For example, the prediction models are such as Cox proportional hazard model, Logistic Regression (LR) model, and Neural Additive Model (NAM). The Cox proportional hazard may output a time-to-event, for example, “the user will not suffer from CHF in the next six months after measuring his ECG signal today”. LR and NAM are classification models, which only inform whether the user will suffer from CHF within a specified period in the future (for example, within one year). The implementation details of the above three models can refer to the following documents:
D. R. Cox, “Partial likelihood,” Biometrika, vol. 62, no. 2, pp. 269-2′76, 1975.
R. E. Wright, “Logistic regression,” Reading and understanding multivariate statistics, pp. 217-244, 1995.
R. Agarwal, N. Frosst, X. Zhang, R. Caruana, and G. E. Hinton, “Neural additive models: Interpretable machine learning with neural nets,” arXiv preprint arXiv:2004.13912, 2020.

In an embodiment of the present disclosure further electrically connects to an environment sensor to detect the environment information around the heart failure predictor 100. The environment sensor is such as an accelerometer or a light sensor. The environment information detected by the environment sensor will serve as a basis for the prediction model circuit 5 to select one of the plurality of prediction models.

In the time-to-event setting, the quantity of interest is the time between the ECG measurement and the CHF event. If the user experiences a CHF event in the observation period, the present disclosure sets a label as the time-to-event and marks the label as uncensored. Otherwise, the label will be set to the user's observation period and be marked as censored. Alternatively, in the classification setting, the quantity of interest is the 1-year CHF outcome. The label for the user is set to 1 if the user had a CHF within one year from the ECG measurement, or set to 0 otherwise.

In another embodiment of the present disclosure, the feature extraction circuit 3 and the prediction model circuit 5 may be integrated into one module and be implemented with multilayer perceptron (MLP) or convolution neural network (CNN). For example, the present disclosure may use a MLP module to receive the clean ECG signal and directly output a prediction regarding whether the user will suffer from CHF within one year. However, said another embodiment lacks of the interpretable feature vector compared to the previous embodiment.

Prediction models adopted by the prediction model circuit 5 are generated by training with a large amount of data. The present disclosure collects ECG signals from a large number of users and the time of heart failure events of these users, and splits the data into three sets for the prediction model's training, validation, and testing. These sets do not share records from the same user. The present disclosure trains the models on the training set and uses the validation set to choose hyper-parameters and sets a threshold for the discrete binary prediction. The present disclosure evaluates the performance of the models on the test set using three metrics: index of concordance (c-index) using the time-to-event labels, Area Under the Curve (AUC) and balanced accuracy (average sensitivity and specificity) using the binary labels. Regardless of the type of labels a model was trained on, the present disclosure can always compute these three metrics since all the models predict a risk estimate. Note that both AUC and balanced accuracy are well suited for managing the imbalance class case.

Performance results are shown in the table below.

| | | | BALANCED |
|---|---|---|---|
| Model | C-INDEX | AUC | ACCURACY |
| baseline | 0.718 | 0.762 | 0.713 |
| Cox | 0.783 | 0.817 | 0.743 |
| LR | 0.788 | 0.809 | 0.743 |
| NAM | 0.798 | 0.821 | 0.752 |
| (24 h) NAM | 0.810 | 0.834 | 0.763 |

From the table, the PCA features considerably improve the baseline's performance: +0.065 in C-index, +0.055 in AUC, and +0.030 in balance accuracy. The NAM model is the interpretable model with the best performance. Even though the NAM model is not trained on time-to-event data, it has a higher C-index than the Cox model. This may be because the features' effect on the outcome is not linear.

Further, the present disclosure quantifies the performance drop of the short 30-second signal compared with the 24-hour signal: −0.012 in C-index, −0.013 in AUC, −0.011 in balanced accuracy. The result shows that the short 30-second signal is very effective in its CHF predictive power.

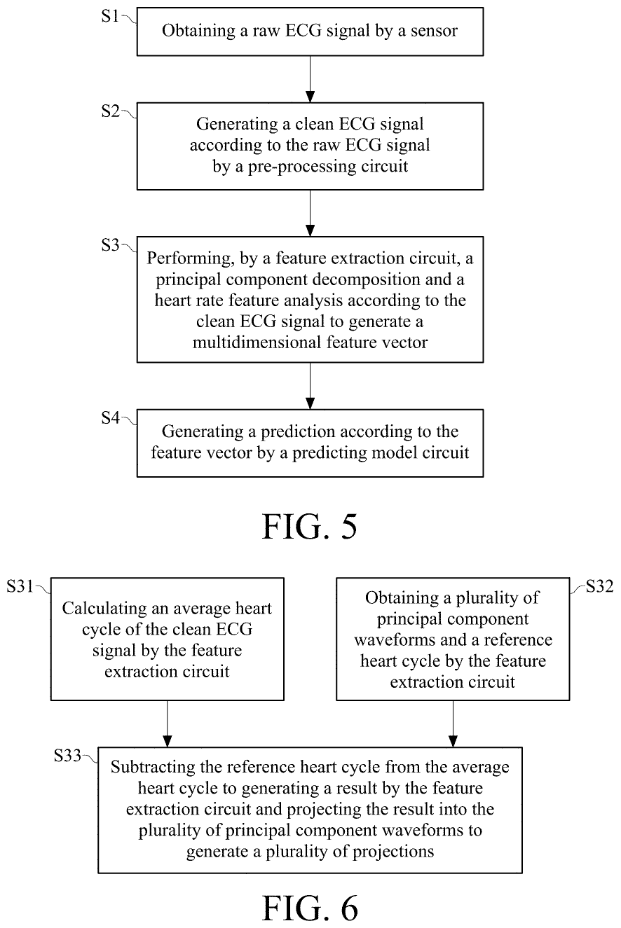

FIG. 5 is a flow chart of the heart failure predicting method according to an embodiment of the present disclosure. Step S1 shows that “obtaining a raw ECG signal by a sensor”. Step S2 shows that “generating a clean ECG signal according to the raw ECG signal by a pre-processing circuit”. Step S3 shows that “performing, by a feature extraction circuit, a principal component decomposition and a heart rate feature analysis according to the clean ECG signal to generate a multidimensional feature vector”. Step S4 shows that “generating a prediction according to the feature vector by a predicting model circuit”. Details of every step has been described in previous paragraphs and will not be repeated here.

FIG. 6 is a detailed flowchart of “principal component analysis” in step S3 of FIG. 5. Step S31 shows that “calculating an average heart cycle of the clean ECG signal by the feature extraction circuit”. Step S32 shows that “obtaining a plurality of principal component waveforms and a reference heart cycle by the feature extraction circuit”. It should be noticed that the present disclosure does not limit the order performing steps S31 and S32. Step S33 shows that “subtracting the reference heart cycle from the average heart cycle to generating a result by the feature extraction circuit and projecting the result into the plurality of principal component waveforms to generate a plurality of projections”. Details of every step has been described in previous paragraphs and will not be repeated here.

In view of the above description, the heart failure predictor and a heart failure predicting method proposed in the present disclosure uses the “shape” feature, that is extracted from a time-series data, to predict the CHF together with HR and HRV. The present disclosure may generate an interpretable feature vector so that a doctor may explain the cause of heart failure to the patient based on the clinical symptoms corresponding to the feature vector. Because the feature extraction circuit of the present disclosure uses the principal component analysis technique, the feature vector includes the shape feature of the ECG signal. Using principal component analysis also improves the accuracy of the present disclosure in predicting heart failure. The present disclosure can generate a prediction result for the long-term future (several months to several years) by collecting one's ECG signal for only a short period of time (for example, 30 seconds).

What is claimed is:

1. A heart failure predictor comprises:
a pre-processing circuit configured to electrically connect to an external sensor to receive a raw electrocardiogram (ECG) signal and configured to filter a noise of the raw ECG signal to generate a clean ECG signal;
a feature extraction circuit electrically connecting to the pre-processing circuit, wherein the feature extraction circuit calculates a plurality of heart rate features according to the clean ECG signal, generates a plurality of shape features according to a plurality of principal component waveforms and the clean ECG signal, and concatenates the plurality of heart rate features and shape features to generate a feature vector; and
a prediction model circuit electrically connecting to the feature extraction circuit and generating a prediction according to the feature vector; wherein
the prediction is configured to indicate whether the heart failure will happen in a specified period.

2. The heart failure predictor of claim 1, wherein the plurality of principal component waveforms are calculated by a processor performing a principal component analysis procedure according to a plurality of historical ECG signals.

3. The heart failure predictor of claim 1, wherein the prediction model circuit comprises a plurality of prediction models, and the plurality of prediction models includes Cox proportional hazard model, logistic regression model and neural additive model.

4. A heart failure predicting method comprising:
obtaining a raw electrocardiogram (ECG) signal by a sensor;
generating a clean ECG signal according to the raw ECG signal by a pre-processing circuit;
performing, by a feature extraction circuit, a principal component decomposition and a heart rate feature analysis according to the clean ECG signal to generate a feature vector with a plurality of features; and
generating a prediction to indicate whether the heart failure will happen in a specified period according to the feature vector by a predicting model circuit.

5. The heart failure predicting method of claim 4, performing the principal component decomposition by the feature extraction circuit comprising:
calculating an average heart cycle of the clean ECG signal by the feature extraction circuit;
obtaining a plurality of principal component waveforms and a reference heart cycle by the feature extraction circuit;
subtracting the reference heart cycle from the average heart cycle to generating a result by the feature extraction circuit and projecting the result into the plurality of principal component waveforms to generate a plurality of projections; wherein
the plurality of projections are a part of the plurality of features of the feature vector;
the plurality of principal component waveforms are generated by a processor performing a principal component analysis procedure according to a plurality of historical ECG signals.

6. The heart failure predicting method of claim 4, wherein the prediction model circuit comprises a plurality of prediction models, and the plurality of prediction models includes Cox proportional hazard model, logistic regression model and neural additive model.
